(19) United States
(12) Patent Application Publication
(10) Pub. No.: US 2004/0091462 A1
(43) Pub. Date: May 13, 2004

(54) Composition for the carrying and delivery of bone growth inducing material and methods for producing and applying the composition

(76) Inventors: Steve T. Lin, Gainesville, FL (US); Luis Z. Avila, Arlington, MA (US); Arthur J. Coury, Boston, MA (US); Hidegard M. Kramer, Westport, CT (US); Laurence A. Roth, Windham, NH (US); Rebecca Roberts, High Springs, FL (US); Michael Kurt Sly, Gainesville, FL (US)

Correspondence Address: GREENBERG TRAURIG, LLP, 885 3RD AVENUE, NEW YORK, NY, 10022, US

(21) Appl. No.: 10/645,744

(22) Filed: Aug. 20, 2003

Related U.S. Application Data
(60) Provisional application No. 60/404,895, filed on Aug. 20, 2002.

(51) Int. Cl. A61K 45/00; A61K 9/14

(52) U.S. Cl. 424/93.7; 424/486

(57) ABSTRACT

Various embodiments of the present invention relate to compositions for delivering bone growth inducing material (e.g., to viable bone and/or other skeletal tissues to repair defects and the like). More particularly, various embodiments of the present invention relate to delivery mechanisms for an osteotherapeutic material (e.g., osteoinductive and/or osteoconductive materials), including (but not limited to) demineralized bone matrix (“DBM”) and cortical-cancellous bone chips (“CCC”). Certain compositions according to various embodiments of the present invention may comprise mixtures of a physiologically acceptable biodegradable carrier, an osteoinductive material, and/or an osteoconductive material (e.g., DBM and CCC). The compositions may thus be applied (for example, to defective bone tissue and/or other viable tissue) to induce formation of new bone. Other embodiments of the present invention relate to the preparation of compositions and methods of using such compositions.

### CROSS-REFERENCE TO RELATED APPLICATIONS

This application claims the benefit under 35 U.S.C. 119(e) of U.S. Provisional Application Serial No. 60/404,895, filed Aug. 20, 2002.

### FIELD OF THE INVENTION

Various embodiments of the present invention relate to compositions for delivering osteotherapeutic material (e.g., to viable bone and/or other skeletal tissues to repair defects and the like). More particularly, various embodiments of the present invention relate to delivery mechanisms for an osteotherapeutic material (e.g., osteoinductive and/or osteoconductive materials), including (but not limited to) demineralized bone matrix (“DBM”) and cortical-cancellous bone chips (“CCC”). Certain compositions according to various embodiments of the present invention may comprise mixtures of a physiologically acceptable biodegradable carrier, an osteoinductive material, and/or an osteoconductive material (e.g., DBM and CCC). The compositions may thus be applied (for example, to defective bone tissue and/or other viable tissue) to promote formation of new bone. Other embodiments of the present invention relate to the preparation of compositions and methods of using such compositions.

For the purposes of the present application the term “osteotherapeutic material” (or “osteotherapeutic factor”) is intended to refer to a material that promotes bone growth. Osteotherapeutic materials, or factors, include (but are not limited to) osteoinductive material, osteoconductive, osteogenic and osteopromotive material. Further, osteotherapeutic materials, or factors, include (but are not limited to): bone morphogenic protein (“BMP”) such as BMP 2, BMP 4 and BMP 7 (OP1); DBM, platelet-derived growth factor (“PDGF”); insulin-like growth factors I and II (“IGF-I”, “IGF-II”); fibroblast growth factors (“FGF's”); transforming growth factor beta (“TGF-β”); platelet rich plasma (PRP); vescular endothelial growth factor (VEGF); growth hormones; small peptides; genes; stem cells, autologous bone, allogenic bone, bone marrow, biopolymers and bioceramics.

Further, for the purposes of the present application the term “osteoinductor”(or “osteoinductive material”) is intended to refer to a material that has the capability of inducing ectopic bone formation. Osteoinductive materials include (but are not limited to): DBM; BMP 2; BMP 4; and BMP 7.

Further still, for the purposes of the present application the term “osteoconductor” (or “osteoconductive material”) is intended to refer to a material that does not have the capability of ectopic bone formation, but provides the surface for the osteoblast cells to adhere, proliferate, and/or synthesize new bone. Osteoconductive materials include (but are not limited to): CCC; hydroxyapatite (“HA”); tricalcium phosphate (“TCP”); mixtures of HA/TCP; other calcium phosphates; calcium carbonate; calcium sulfate; collogen; and DBM.

Further still, for the purposes of the present application the term “osteogenic factor” (or “osteogenic material”) is intended to refer to a material that supplies and supports the growth of bone healing cells. Osteogenic materials include (but are not limited to): autogenous cancellous bone, bone marrow, periosteum, and stem cells.

Further still, for the purposes of the present application the term “osteopromoter” (or “osteopromotive material”) is intended to refer to a material that enhances or accelerates the natural cascade of bone repair. Osteogenic materials include (but are not limited to): PRP, FGF's, TGF-β, PDGF, VEGF.

Further still, for the purposes of the present application the term “patient” is intended to refer to any animal (e.g., human, mammal, vertebrate) into which a composition, carrier, and/or osteotherapeutic material according to the present invention is implanted.

### BACKGROUND OF THE INVENTION

Compounds purporting to facilitate the repair of bone defects have been previously disclosed. Likewise, compositions that may function as carriers for the delivery of drugs and other therapeutic agents (which carriers are macromers containing a central block of poly(ethylene glycol)) are likewise previously disclosed.

### BRIEF DESCRIPTION OF THE DRAWINGS

FIG. 1 is a bar graph showing bone induction score for DBM control and as a function of concentration of DBM;

FIG. 2a shows the histology of implanted macromer alone;

FIG. 2b shows the histology of TBI DBM in macromer;

FIG. 2c shows the histology of 30% DBM in macromer;

FIG. 3 is a bar graph showing mechanical test results; and

FIGS. 4a-4e show results related to Example 19, discussed below.

Among those benefits and improvements that have been disclosed, other objects and advantages of this invention will become apparent from the following description taken in conjunction with the accompanying figures. The figures constitute a part of this specification and include illustrative embodiments of the present invention and illustrate various objects and features thereof.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

DBM is the protein component of bone. It is prepared from donated bone tissue by first grinding the cortical bone to desired particle size, then removing minerals from the bone particles in hydrochloric acid, and finally lyophilizing demineralized particles to eliminate water.

Cortical cancellous bone chips are a mixture of cortical and cancellous bone particles that are milled or grinded from cortical and cancellous bone.

Demineralized allograft bone powder is typically available in a lyophilized or freeze dried and sterile form to provide for extended shelf life. The demineralized bone component of the composition herein is a known type of pulverized or powdered material and is prepared in accordance with known procedures. It should be understood that the term “demineralized bone matrix” includes bone particles of a wide range of average particle size ranging from relatively fine powders to coarse grains and even larger chips. So, for example (which example is intended to be illustrative and not restrictive), the bone powder present in the composition of this invention may range in average particle size from about 100 to about 1,200 μm or from about 125 to 850 μm.

In general, human allogenic bone tissue may be preferred as the source of the bone powder.

The macromers that are employed as carriers may include at least one water-soluble block, at least one biodegradable block, and at least one polymerizable group. At least one biodegradable block may contain a carbonate or ester group. To obtain a biodegradable material after polymerization, each polymerizable group may need to be separated from any other polymerizable group on the macromer by at least one biodegradable linkage or group.

In one example (which example is intended to be illustrative and not restrictive) at least a portion of the macromers may contain more than one reactive group and thereby be effective as crosslinkers, so that the macromers may be crosslinked to form a gel. The minimal proportion required will vary with the nature of the macromer and its concentration in solution, and the proportion of crosslinker in the macromer solution may be as high as 100% of the macromer solution.

Since in certain homolytic (free radical) polymerization reactions each polymerizable group will polymerize into a chain, crosslinked hydrogels may be produced using only slightly more than one reactive group per macromer (i.e., about 1.02 polymerizable groups on average). However, higher percentages may be used, and excellent gels may be obtained in polymer mixtures in which most or all of the molecules have two or more reactive double bonds. Poloxamines, an example of a water-soluble block, have four arms and thus may readily be modified to include four polymerizable groups.

As used herein, a “biocompatible” material is one which stimulates (at worst) only a mild, often transient, implantation response, as opposed to a severe or escalating response.

As used herein, a “biodegradable” material is one which decomposes under normal in vivo physiological conditions into components which may be metabolized and/or excreted.

As used herein, a “block” is a region of a macromer differing in subunit composition from neighboring regions. Blocks will typically contain multiple subunits, up to about one thousand subunits or less for non-degradable materials, and without an upper limit for degradable materials. In the lower limit, the size of a block typically depends on its function; the minimum size is that which is sufficient to allow the function to be performed. In the case of a block conferring water-solubility on the macromer, for example, this may be 400 daltons or more, 600 daltons or more, at least 1000 daltons, or be in the range of 2000 to 40,000 daltons. For degradable linkages, the minimum block size is a single linkage of the appropriate degradability for the function. In one example (which example is intended to be illustrative and not restrictive) the block size may be two to forty groups or three to twenty groups. The reactive groups may be considered as a block for some purposes; the typical number of units in such a block is one, but may be, for example two to five.

As used herein, a carbonate is a functional group with the structure

—O—C(O)—O—. The carbonate starting material may be cyclic, such as trimethylene carbonate (TMC), or may be linear, such as dimethylcarbonate (CH₃O—C(O)—OCH₃). After incorporation into the polymerizable macromer, the carbonate may be present at least in part as R—O—C(═O)—O—R′, where R and R′ are other components of the macromer.

As used herein, an ester is a repeating unit with the structure—O—C(O)—R—O—, where R is a straight, branched or cyclic alkyl group.

As used herein, a hydrogel is a substance formed when an organic polymer (natural or synthetic) is cross-linked via covalent, ionic, or hydrogen bonds to create a three-dimensional open-lattice structure which entraps water molecules to form a gel.

As used herein, “water-soluble” is defined as a solubility of at least one gram/liter in an aqueous solution at a temperature in the range of about 0° C. and 50° C. Aqueous solutions may include small amounts of water-soluble organic solvents, such as dimethylsulfoxide, dimethylformamide, alcohols, acetone, and/or glymes.

Types of Block Macromers

In general terms, the macromers may, in one example (which example is intended to be illustrative and not restrictive) be block macromers that comprise a biodegradable block, a water-soluble block, and at least one polymerizable group. In one example (which example is intended to be illustrative and not restrictive) the macromers may comprise at least 1.02 polymerizable groups on average or may include at least two polymerizable groups per macromer, on average. Average numbers of polymerizable groups may be obtained, for example, by blending macromers with different amounts of polymerizable groups.

The individual blocks may be arranged to form different types of block macromers, including di-block, tri-block, and multi-block macromers. The polymerizable groups may be attached directly to biodegradable blocks or indirectly via water-soluble nondegradable blocks, and may be attached so that the polymerizable groups are separated from each other by a biodegradable block. For example (which example is intended to be illustrative and not restrictive), if the macromer contains a water-soluble block coupled to a biodegradable block, one polymerizable group may be attached to the water-soluble block and another attached to the biodegradable block. Both polymerizable groups may be linked to the water-soluble block by at least one degradable linkage.

The di-block macromers may include a water-soluble block linked to a biodegradable block, with one or both ends capped with a polymerizable group. The tri-block macromers may include a central water-soluble block and outside biodegradable blocks, with one or both ends capped with a polymerizable group. Alternatively, the central block may be a biodegradable block, and the outer blocks may be water-soluble. The multiblock macromers may include one or more of the water-soluble blocks and biocompatible blocks coupled together in a linear fashion. Alternatively, the multi-block macromers may be brush, comb, dendritic or star copolymers. If the backbone is formed of a water-soluble block, at least one of the branches or grafts attached to the backbone may be a biodegradable block. Alternatively, if the backbone is formed of a biodegradable block, at least one of the branches or grafts attached to the backbone may be a water-soluble block, unless the biodegradable block is also water-soluble. In another embodiment, a multifunctional compound, such as a polyol, may be coupled to multiple polymeric blocks, at least one of which may be water-soluble and at least one of which may be biodegradable.

In general, any formulation of the macromer which is intended to be biodegradable may need to be constructed so that each polymerizable group is separated from each other polymerizable group by one or more linkages which are biodegradable. Non-biodegradable materials may not necessarily be subject to this constraint.

Those skilled in the art will recognize that the individual blocks may have uniform compositions, or may have a range of molecular weights, and may be combinations of relatively short chains or individual species which confer specifically desired properties on the final hydrogel, while retaining the required characteristics of the macromer. The lengths of blocks referred to herein may vary from single units (e.g., in the biodegradable portions) to a few repeating units such as oligomeric blocks to yet many repeating units such as in polymeric blocks, subject to the constraint of preserving the overall water-solubility of the macromer.

In the discussion below and the examples, macromers are often designated by a code of the form xxKZn wherein xx are the digits that represent the molecular weight of the backbone polymer, which is polyethylene glycol (“PEG”) unless otherwise stated, and K the unit in thousands of Daltons; followed by a letter which designates the biodegradable linkage, shown here as Z, where Z may be one or more of L, G, D, C or T, wherein L is for lactic acid, G is for glycolic acid, D is for dioxanone, C is for caprolactone, T is for trimethylene carbonate, and n is the average number of degradable groups in the block. The molecules are terminated with acrylic ester groups, unless otherwise stated. This is sometimes also indicated by the suffix A2.

While the biodegradable groups may be, for example (which example is intended to be illustrative and not restrictive) (in addition to carbonate or ester): hydroxy acids, orthoesters, anhydrides, or other synthetic or semisynthetic degradable linkages, natural materials may be used in the biodegradable sections when their degree of degradability is sufficient for the intended use of the macromer. Such biodegradable groups may comprise, for example (which example is intended to be illustrative and not restrictive), natural or unnatural amino acids, carbohydrate residues, and other natural linkages. Biodegradation time may be controlled by the local availability of enzymes hydrolyzing such linkages. The availability of such enzymes may be ascertained from the art or by routine experimentation.

Water Soluble Regions

Suitable water-soluble polymeric blocks may include those prepared from poly(ethylene glycol), poly(ethylene oxide), partially or fully hydrolyzed poly(vinyl alcohol), poly(vinylpyrrolidone), poly(ethyloxazoline), poly(ethylene oxide)-copoly(propylene oxide) block copolymers (poloxamers and meroxapols), poloxamines, carboxymethyl cellulose, hydroxyalkylated celluloses such as hydroxyethyl cellulose and methylhydroxypropyl cellulose, polypeptides, polynucleotides, polysaccharides or carbohydrates such as Ficoll®. polysucrose, hyaluronic acid, dextran, chondroitin sulfate, heparin, or alginate, and proteins such as gelatin, collagen, albumin, or ovalbumin.

The soluble polymer blocks may be intrinsically biodegradable or may be poorly biodegradable or effectively non-biodegradable in the body. In the latter two cases, the soluble blocks may be of sufficiently low molecular weight to allow excretion. The maximum molecular weight to allow excretion in human beings (or other species in which use is intended) will vary with polymer type, but will often be about 40,000 daltons or below. Water-soluble natural polymers and synthetic equivalents or derivatives, including polypeptides, polynucleotides, and degradable polysaccharides, may be used.

The water-soluble blocks may be a single block with a molecular weight, for example (which example is intended to be illustrative and not restrictive), of at least 600 Daltons, 2000 or more Daltons, or at least 3000 Daltons. Alternatively, the water-soluble blocks may be two or more water-soluble blocks which are joined by other groups. Such joining groups may include biodegradable linkages, polymerizable linkages, or both. For example (which example is intended to be illustrative and not restrictive), an unsaturated dicarboxylic acid, such as maleic, fumaric, or aconitic acid, may be esterified with degradable groups as described below, and such linking groups may be conjugated at one or both ends with hydrophilic groups such as polyethylene glycols. In another embodiment, two or more PEG molecules may be joined by biodegradable linkages including carbonate linkages, and subsequently be end-capped with polymerizable groups.

Biodegradable Blocks

The biodegradable blocks may be hydrolyzable under in vivo conditions. At least one biodegradable region may be a carbonate or ester linkage. Additional biodegradable polymeric blocks may include polymers and oligomers of hydroxy acids or other biologically degradable polymers that yield materials that are non-toxic or present as normal metabolites in the body. Usable poly(hydroxy acid)s are poly(glycolic acid), poly(DL-lactic acid) and poly(L-lactic acid). Other useful materials include, polycarbonates such as poly(trimethylene carbonate), poly(amino acids), poly(anhydrides), poly(orthoesters), and poly(phosphoesters). Polylactones such as poly(epsilon-caprolactone), poly(delta-valerolactone), poly(gamma-butyrolactone) and poly (beta-hydroxybutyrate), for example (which example is intended to be illustrative and not restrictive), are also useful.

Biodegradable regions may be constructed from monomers, oligomers and/or polymers using linkages susceptible to biodegradation, such as ester, peptide, anhydride, orthoester, and phosphoester bonds.

By varying the total amount of biodegradable groups, and selecting the ratio between the number of carbonate or ester linkages (which are relatively slow to hydrolyze) and of lower hydroxy acid linkages (especially glycolide or lactide, which hydrolyze relatively rapidly), the degradation time of hydrogels formed from the macromers may be controlled.

Carbonates

Any desired carbonate may be used to make the macromers. Such carbonates may include (but not be limited to) aliphatic carbonates (e.g., for maximum biocompatibility). For example (which example is intended to be illustrative and not restrictive), trimethylene carbonate and dimethyl carbonate are examples of aliphatic carbonates. Lower dialkyl carbonates are joined to backbone polymers by removal by distillation of alcohols formed by equilibration of dialkyl carbonates with hydroxyl groups of the polymer.

Other useful carbonates are the cyclic carbonates, which may react with hydroxy-terminated polymers without release of water. Suitable cyclic carbonates include ethylene carbonate (1,3-dioxolan-2-one), propylene carbonate (4-methyl-1,3-dioxolan-2-one), trimethylene carbonate (1,3-dioxan-2-one) and tetramethylene carbonate (1,3-dioxepan-2-one). Under some reaction conditions, it is possible that orthocarbonates may react to give carbonates, or that carbonates may react with polyols via orthocarbonate intermediates, as described in Timberlake et al., U.S. Pat. No. 4,330,481. Thus, certain orthocarbonates, particularly dicyclic orthocarbonates, may be suitable starting materials for forming the carbonate-linked macromers.

Alternatively, suitable diols or polyols, including backbone polymers, may be activated with phosgene to form chloroformates, as is described in the art, and these active compounds may be mixed with backbone polymers containing suitable groups, such as hydroxyl groups, to form macromers containing carbonate linkages.

All of these materials are “carbonates” as used herein.

Suitable dioxanones include dioxanone (p-dioxanone; 1,4-dioxan-2-one; 2-keto-1,4-dioxane), and the closely related materials 1,4-dioxolan-2-one, 1,4-dioxepan-2-one and 1,5-dioxepan-2-one. Lower alkyl, for example (which example is intended to be illustrative and not restrictive) C1-C4 alkyl, derivatives of these compounds are also contemplated, such as 2-methyl p-dioxanone (cyclic O-hydroxyethyl ether of lactic acid).

Polymerizable Groups

As used in the present application, a “polymerizable group” contains: (a) a functional group that reacts spontaneously or under the influence of light, heat or other activating conditions or reagents, to form a covalent polymeric structure that binds the macromer strands to one another (hereinafter sometimes referred to as a “macromer-macromer functional group”); and/or (b) a reactive functional group for converting a solution of the macromer into a gel.

When the macromer contains two or more macromer-macromer functional groups, the polymeric structures formed by these groups form crosslinks between the macromer strands leading to a three dimensional network that is a non-fluid gel.

Suitable macromer-macromer functional groups include ethylenic groups (such as vinyl, allyl, acryloyl, cinnamoyl, fumaroyl, styryl), epoxides, lactones (such as lactide, glycolide, caprolactone, valerolactone, dioxanone), lactams (beta-lactams, gammalactams and delta-lactams, gamma-butyrolactam, delta-caprolactam).

A reactive functional group is a group which reacts under nucleophilic, electrophilic, oxidative or radical conditions with a chemical partner to form a covalent bound with that chemical partner in a coupling reaction.

Suitable reactive functional groups include activated esters (such as N-hydroxysuccinimide ester), electrophilic carbon centers (such as tosylates and mesylates), conjugated ethylenic groups (such as acryloyl, methacryloyl), isocyanates, isothiocyanates, oxirane, aziridines, cyclic imides (such as maleimide), sulfhydryls. Suitable chemical partners includes amines, alcohols, thiols.

In some embodiments, the reactive functional group and the chemical partner may be present on different macromer strands and the components may be mixed when the gellation of the solution is desired. In other embodiments, the reactive functional group and the chemical partner may be both present on the same macromer strand, and activating conditions such as oxidative, acidic, radical and the like may be further required to effect gellation.

The polymerizable groups may be located at one or more ends of the macromer or the polymerizable groups may be located within the macromer.

Polymerization may be initiated by any convenient reaction, including, but not limited to, photopolymerization, chemical or thermal free-radical polymerization, redox reactions, cationic polymerization, and chemical reaction of active groups (such as isocyanates, for example.) Polymerization may be initiated using photoinitiators. Photoinitiators that generate a free radical or a cation on exposure to UV light are well known to those of skill in the art. Free-radicals may also be formed in a relatively mild manner from photon absorption of certain dyes and chemical compounds. The polymerizable groups maybe polymerizable by free radical polymerization. Usable polymerizable groups include, but are not limited to, acrylates, diacrylates, oligoacrylates, methacrylates, dimethacrylates, oligomethacrylates, cinnamates, dicinnamates, oligocinnamates, and other biologically acceptable photopolymerizable groups.

These groups may be polymerized using photoinitiators that generate free radicals upon exposure to light, including UV (ultraviolet) and IR (infrared) light, long-wavelength ultraviolet light (LWUV) or visible light. Of note, LWUV and visible light may cause less damage to tissue and other biological materials than short-wave UV light. Useful photoinitiators are those which may be used to initiate polymerization of the macromers without cytotoxicity and within a short time frame (e.g., minutes or seconds).

Exposure of dyes (e.g., in combination with co-catalysts such as amine) to light, (e.g., visible or LWUV light), may generate free radicals. Light absorption by the dye may cause the dye to assume a triplet state, and the triplet state subsequently reacts with the amine to form a free radical which initiates polymerization, either directly or via a suitable electron transfer reagent or co-catalyst, such as an amine. Polymerization may be initiated by irradiation with light at a wavelength of between about 200-1200 nm for example, in the long wavelength ultraviolet range or visible range, for example, at about 320 nm or higher, for example, or between about 365 and 550 nm, for example.

Numerous dyes may be used for photopolymerization. Suitable dyes are well known to those of skill in the art. Such dyes may include, but are not limited to, erythrosin, phloxime, rose bengal, thionine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, other acetophenone derivatives, and camphorquinone. Suitable coinititators may include, but are not limited to, amines such as N-methyl diethanolamine, N,N-dimethyl benzylamine, triethanolamine, triethylamine, dibenzyl amine, N-benzylethanolamine, N-isopropyl benzylamine. Triethanolamine may be used as a coinitiator.

Suitable chemical, thermal and redox systems may initiate the polymerization of unsaturated groups by generation of free radicals in the initiator molecules, followed by transfer of these free radicals to the unsaturated groups to initiate a chain reaction. Peroxides and other peroxygen compounds are well-known in this regard, and may be considered as chemical or thermal initiators. Azobisbutyronitrile is a chemical initiator. A combination of a transition metal, especially iron, with a peroxygen and possibly a stabilizing agent such as glucuronic acid allows generation of free radicals to initiate polymerization by a cycling redox reaction.

Combinations of chemical or redox systems with photoinitiated systems have been demonstrated to be effective in WO 96/29370, and may be used as an initiation system for many applications of the macromers of the present invention. The teachings of WO 96/29370 are incorporated herein by reference.

It is also possible to use the macromers with other types of linking reactions. For example (which example is intended to be illustrative and not restrictive), a macromer may be constructed with amine termination, with the amine considered as a nucleophilic group; and another macromer could be constructed with isocyanate termination, with the isocyanate as the reactive functional group. On mixing, the materials may spontaneously react to form a gel. Alternatively, an isocyanate-terminated macromer may be polymerized and crosslinked with a mixture of diamines and triamines. Such a reaction may be more difficult to control than a photoinitiated reaction, but may be used for high volume extracorporeal production of gels for implantation (e.g., perhaps as drug delivery systems). Other pairs of reactants may include, but not be limited to, maleimides with amines or sulfhydryls, or oxiranes with amines, sulfhydryls or hydroxyls.

Preferred Macromers

The macromers may contain, for example (which example is intended to be illustrative and not restrictive) between about 0.3% and 20% by weight of carbonate residues or ester residues, between about 0.5% and 15% carbonate or ester residues, or about 1% to 5% carbonate or ester residues. In those embodiments where hydroxy acid residues are desired, the macromer may contain, for example (which example is intended to be illustrative and not restrictive), between about 0.1 and 10 residues per residue of carbonate or ester, between about 0.2 and 5, or one or more such residue per macromer.

In another example (which example is intended to be illustrative and not restrictive), the macromer may include a core, an extension on each end of the core, and an end cap on each extension. The core may be a hydrophilic polymer or oligomer; each extension may be a biodegradable oligomer comprising one or more carbonate or ester linkage; and each end cap may comprise one or more functional groups capable of cross-linking the macromers. The core may include hydrophilic poly(ethylene glycol) oligomers with a molecular weight between about 400 and 40,000 Da; each extension may include 1 to 10 residues selected from carbonate and ester, and optionally further included between one and five hydroxyacid residues (e.g., alpha-hydroxy acid residues); wherein the total of all residues in the extensions is sufficiently small to preserve water-solubility of the macromer (being typically less than about 20% of the weight of the macromer (e.g., 10% or less)).

Each end cap may include a polymerizable group. Such groups may be free-radical (homolytically) polymerizable. Such groups may be ethylenically-unsaturated (i.e., containing carbon-carbon double bonds), with a molecular weight between about 50 and 300 Da (for example (which example is intended to be illustrative and not restrictive)), which are capable of cross-linking and/or polymerizing the macromers. Another example (which example is intended to be illustrative and not restrictive) may incorporate a core consisting of poly(ethylene glycol) oligomers of molecular weight about 25,000 Da; extensions including polycarbonate or poly(dioxanone) oligomers with a molecular weight of about 200 to 1000 D, alone or in combination with extensions formed of hydroxy acid oligomers; and end caps consisting of acrylate moieties (which are about 55 Da molecular weight).

Macromer Synthesis

The macromers may be synthesized using means well known to those of skill in the art. General synthetic methods are found in the literature, for example in U.S. Pat. No. 5,410,016 to Hubbell et al., U.S. Pat. No. 4,243,775 to Rosensaft et al., and U.S. Pat. No. 4,526,938 to Churchill et al. These references are incorporated herein by reference.

For example (which example is intended to be illustrative and not restrictive), a polyethylene glycol backbone may be reacted with trimethylene carbonate (TMC) or a similar carbonate in the presence of a Lewis acid catalyst, such as stannous octoate, to form a TMC-polyethylene glycol terpolymer. The TMC-PEG polymer may optionally be further derivatized with additional degradable groups, such as lactate groups. The terminal hydroxyl groups may then be reacted with acryloyl chloride in the presence of a tertiary amine to end-cap the polymer with acrylate end-groups. Similar coupling chemistry may be employed for macromers containing other water-soluble blocks, biodegradable blocks, and/or polymerizable groups (particularly those containing hydroxyl groups).

When polyethylene glycol is reacted with TMC and a hydroxy acid in the presence of an acidic catalyst, the reaction may be either simultaneous or sequential. As shown in the examples below, the simultaneous reaction may produce an at least partially random copolymer of the three components. Sequential addition of a hydroxy acid after reaction of the PEG with the TMC may tend to produce an inner block of TMC and one or more blocks of PEGs, which will statistically contain more than one PEG residue linked by linkages derived from TMC, with hydroxy acid largely at the ends of the (TMC, PEG) region. There is a tendency for TMC and other carbonate groups to rearrange by “back-biting” during synthesis, which is why multiple PEG molecules may become incorporated in the same macromer. When the hydroxy acid contains a secondary hydroxyl, as in lactic acid, then the tendency towards rearrangement may be reduced.

In principle, the degradable blocks or regions may be separately synthesized and then coupled to the backbone regions. In practice, this more complex reaction does not appear to be required to obtain useful materials.

Sequential Addition

In one example (which example is intended to be illustrative and not restrictive), sequential addition of biodegradable groups to a carbonate-containing macromer may be used to enhance biodegradability of the macromer after capping with reactive end groups.

Upon reaction of, for example (which example is intended to be illustrative and not restrictive), trimethylene carbonate (TMC) with polyethylene glycol (PEG), the TMC linkages in the resulting block polymers have been shown to form end linked species of PEG, resulting in segmented polymers, i.e. PEG units coupled by one or more adjacent TMC linkages. The length of the TMC segments may vary, and is believed to exhibit a statistical distribution. Coupling may also be accomplished via the carbonate subunit of TMC. It is believed that these segmented PEG/TMC block polymers form as a result of transesterification reactions involving the carbonate linkages of the TMC segments during the TMC polymerization process when a PEG diol is used as an initiator. Similar behavior is expected if other polyalkylene glycol initiators were used. The end-linking may begin during the reaction of the TMC with the PEG, and completion of the end linking and attainment of equilibrium is observable by a cessation of increase of the viscosity of the solution.

If the product of this first reaction step is then reacted with a reactive end-capping material, such as acryloyl chloride (for example (which example is intended to be illustrative and not restrictive)), a significant percentage of the macromer end groups may be PEG hydroxyls, resulting in the attachment of the reactive groups directly to one end of a non-biodegradable PEG molecule. Such a reaction of the PEG/TMC segmented block polymers may be prevented by adding additional segments of other hydrolyzable Z units (e.g. lactate, glycolate, 1,4-dioxanone, dioxepanone, caprolactone) on either end of the PEG/TMC segmented block polymer. Some scrambling of the additional segments with the PEG/TMC block polymer is expected, but this may be minimized by using proper reaction conditions. The basic PEG/TMC segmented polymer or the further reacted PEG/TMC/Z segmented terpolymer may then be further reacted to form crosslinkable macromers by affixing reactive end groups (such as acrylates) to provide a macromer with reactive functionality. Subsequent reaction of the end groups in an aqueous environment results in a bioabsorbable hydrogel. Similar segmented structures would be expected if another polyalkylene glycol (PAG) were used, for example, a poloxamer.

The block polymers and macromers may have tailorable solubility and solution viscosity properties. The hydrogels may have tailorable modulus and degradation rate. For a given solution concentration in water, the viscosity is affected by the degree of end linking, the length of the TMC (and other hydrophobic species) segments, and the molecular weight of the starting PAG. The modulus of the hydrogel is affected by the molecular weight between crosslinks. The hydrogel degradation rate may be modified by adding a second, more easily hydrolyzed comonomer (e.g. lactate, glycolate, 1,4-dioxanone) as a segment on the ends of the basic PAG/TMC block polymer prior to adding the crosslinkable end group to form the macromer.

Some of these structures described herein are depicted below. PEG, lactate and acrylate units are used solely for purposes of illustration.

Some Basic Structures:

(CH₂—CH₂—O)ₓ=PEG repeat unit=(PEG)ₓ

(CO—(CH₂)₃—O)_{y}=TMC repeat unit=(TMC)_{y}

(CO—CH(CH₃)—O)_{z}=Lactate repeat unit=(LA)_{z}

—CO—CH═CH₂=Acrylate end group=AA

Segmented PEG/TMC Block Polymer:

HO—(CO—(CH₂)₃—O)_{y}—[(CH₂—CH₂—O)ₓ—(CO—(CH₂)₃—O)_{y}]ₙ—H or HO-(TMC)_{y}-[(PEG)ₓ-(TMC)_{y}]ₙ-H

Segmented PEG/TMC/Lactate Terpolymer:

HO—(CH(CH₃)—CO)_{z}—O—(CO—(CH₂)₃—O)_{y}—[(CH₂—CH₂—O)ₓ—(CO—(CH₂)₃—O)_{y}]ₙ—(CO—CH(CH₃)—O)_{z}—H or HO-(LA)_{z}-(TMC)_{y}-[(PEG)ₓ-(TMC)_{y}]ₙ-(LA)_{z}-H

Segmented PEG/TMC Macromer (acrylated):

CH₂═CH—CO—O—(CO—(CH₂)₃—O)_{y}[(CH₂—CH₂—O)ₓ—(CO—(CH₂)₃—O)_{y}]ₙ—CO—CH═CH₂ or .AA-(TMC)._{y}-[(PEG)ₓ-(TMC)_{y}]ₙ-AA

Segmented PEG/TMC/Lactate Terpolymer Macromer (acrylated):

AA-(LA)_{z}-(TMC)_{y}-[(PEG)ₓ-(TMC)_{y}]ₙ-(LA)_{z}-AA

wherein AA represents an acrylate end group

The applicants have found that one suitable carrier is the FocalSeal®-S sealant, available from Genzyme Corp., Cambridge, Mass., USA. It is the applicants understanding that the FocalSeal®-S sealant is an aqueous solution containing a macromer of PEG, trimethylene carbonate (TMC) and poly(lactic acid), with acrylic ester end groups. As set forth above, the composition may or may not include an initiator, such as a photoinitiator.

Preparation and Use of Composition

In yet another embodiment, the composition may be frozen prior to storage and use, which may improve stability.

In yet another embodiment, the composition may initially be a dry product that is reconstituted, with water or other solution, prior to use. The composition may be dried by air drying or freeze drying if initially manufactured with water. In another embodiment, the composition may be blended dry.

Reconstitution may employ a liquid such as sterile water, saline solution, lactated ringer's solution, etc., in order to regain the consistency of putty. The reconstituting liquid may also include agents that make the putty polymerizable during or after implantation.

The composition may include suitable additives (in effective amounts) in order to improve and/or enhance one or more properties of the composition. Examples of such additives, which is not intended to be a complete list, include those additives which improve the composition's bioactive effect, those which initiate polymerization, those which control the rate of polymerization, those which improve handling of the composition, or those which improve the processing of the composition. For example (which example is intended to be illustrative and not restrictive), adding hyaluronic acid to the composition increases composition viscosity, making it easier to handle. In another example (which example is intended to be illustrative and not restrictive) tert-butanol may be added to improve processing, as this agent improves the freeze-drying procedure.

Therapeutic agents, such as drugs, may also be included in the composition. Other bioactive agents, including but not limited to proteins (e.g., bone morphogenic proteins, gene sequences, and/or stem cells), may be included in the composition.

The composition may also include minerals (e.g., calcium, phosphates, etc.), biological macromolecules (collagen, hyaluronic acid, etc.), and polymerization agents (e.g., photochemical, redox (chemical), etc.). Some additives are best added during manufacture, and some others are best added just prior to implant (e.g. stem cells, gene sequences, etc.).

Polymerization may be performed in the operating room (either on the operating table or on the surgery site itself). The polymerization may also be performed at a remote location (i.e., at the manufacturing site) and processed subsequently.

In another embodiment the composition and/or carrier and/or osteotherapeutic material may take the form of, for example (which example is intended to be illustrative and not restrictive): (a) a powder; (b) a dough or paste; (c) a solid or semi-sold (e.g., any desired shape such as, for example, a flat sheet); and/or (d) granules.

In another embodiment the composition and/or carrier and/or osteotherapeutic material may take the form of, for example (which example is intended to be illustrative and not restrictive): (a) fibers; (b) fabrics (including non-wovens, gauzes); (c) films; and/or (d) monolithics.

In another embodiment the composition and/or carrier and/or osteotherapeutic material (e.g., peptide) may be incorporated by, for example (which example is intended to be illustrative and not restrictive): (a) physical admixture; (b) covalent attachment; (c) ionic attachment; and/or (d) physical interpenetration.

In another embodiment the composition and/or carrier and/or osteotherapeutic material may be used by, for example (which example is intended to be illustrative and not restrictive): (a) mixing with fluid and then implanting; and/or (b) implanting dry (e.g., packing the defect), then hydrating with a fluid.

In another embodiment the composition and/or carrier and/or osteotherapeutic material may be used as a coating or adjuvant to another implant (e.g. spinal cage, screw, knee/hip implant, periodontal implant and/or craniofacial implant).

In another embodiment the composition and/or carrier and/or osteotherapeutic material may be used for growing bone in a heterotopic site (e.g., if the product is used by itself (e.g., without a cage during spinal fusion)).

In another embodiment the composition may be polymerized into a pre-selected shape. The polymerization may take place at a site remote from the operating room (e.g., a site of manufacture) and/or in the operating room before implantation (e.g., immediately prior to placement at the final implant site, that is, polymerization is performed at a table in the operating room) and/or in the body at the actual site of the bone defect (e.g., the composition in the form of a powder may be placed in the bone defect and the composition may pick-up moisture from the environment).

In another embodiment any desired diluents may be used to re-hydrate a preformed hydrogel+DBM+CCC.

In another embodiment the additive to modify at least one of a physical and a chemical aspect of the composition may be selected from the group including, but not limited to: (a) a stabilizer (e.g., to protect the composition from radiation damage); (b) a viscosity enhancing agent; and/or (c) a modifier.

In another embodiment the additive to modify a biological aspect of the composition may be selected from the group including, but not limited to: (a) a therapeutic agent; (b) a bioactive agent; (c) a mineral; (d) one or more biological macromolecules; and/or (e) plasma.

In another embodiment applied radiation may be selected from the group including, but not limited to: visible light, gamma radiation. In another embodiment biological fluid may include (but is not limited to): blood and plasma.

Polymerization may be initiated by photochemical means, by non-photochemical like redox (Fenton chemistry) and/or thermal initiation (peroxide, etc). Photochemical initiators may include, but are not limited to, visible light and UV light sensitive compounds like eosin Y, Irgacure, etc.

The composition may be polymerized into desired shapes like rods, sheets, spheres, discs, fleece, powder, foam, etc. The polymerized composition (if manufactured outside the operating room) may be further dried and then allowed to rehydrate in the time prior to implantation.

During rehydration, the composition may be tailored to give products that slightly swell into place for anchoring purposes. Rehydration might also allow for incorporation of fluids (e.g. blood (e.g., the patient's own blood), stem cells, and/or additional drug or other externally derived agents) immediately prior to implant. Dried products may also exhibit adhesive property during application because of rehydration.

The composition may be applied to defective bone tissue and other viable tissue to induce formation of new bone.

The carrier may be selected from a group of biocompatible, biodegradable, polymerizable and at least substantially water-soluble macromers. The macromers may be block copolymers that include at least one water-soluble block, at least one biodegradable block, and at least one polymerizable group. At least one of the biodegradable blocks may comprise a linkage based on a carbonate or ester group, and the macromers may contain other degradable linkages or groups in addition to carbonate or ester groups.

In one embodiment, the macromers may be polymerized using free radical initiators under the influence of long wavelength ultraviolet light or visible light excitation. Biodegradation occurs at the linkages within the extension oligomers and results in fragments, which are non-toxic and removed from the body in normal physiological processes.

Suitable water-soluble polymeric blocks include those prepared from poly(ethylene glycol), poly(ethylene oxide), among others enumerated herein.

At least one biodegradable region may be a carbonate or ester linkage. Biodegradable polymeric blocks may include polymers and oligomers of hydroxy acids or other biologically degradable polymers that yield materials that are non-toxic or present as normal metabolites in the body. Such poly(hydroxy acids) are poly(glycolic acid), poly(DL-lactic acid) and poly(L-lactic acid).

Usable carbonates are aliphatic carbonates (e.g., for maximum biocompatibility). For example (which example is intended to be illustrative and not restrictive), trimethylene carbonate and dimethyl carbonate are examples of aliphatic carbonates.

In one embodiment, the composition may include the macromer, an osteoinductive material, and an osteoconductive material. In another embodiment, the osteoconductive material and the osteoinductive material are distinct components. In another embodiment, the osteoinductive material and the osteoconductive material are DBM and CCC. In another embodiment, the macromer is polymerized, either in production of the carrier or after delivery in situ. In this case, polymerization may be initiated by any convenient reaction, including photopolymerization, chemical or thermal free-radical polymerization, redox reactions, cationic polymerization, and chemical reaction of active groups (such as isocyanates, for example). In one example (which example is intended to be illustrative and not restrictive) polymerization may be initiated using photoinitiators, such as eosin Y, which may be further incorporated into compositions along with the macromer, osteoinductor, and osteoconductor.

In another embodiment, the osteoinductive material and/or osteoconductive material may be added to the macromer, and a photoinitiator may be further included in the mixture. The mixture may form a viscous and cohesive mass that results in an injectable and moldable putty. The composition may be stored at about −40° C. and sealed from the light to maintain its stability and prevent shelf-degradation of the putty. When used in surgery, the allograft putty may convert to a semisolid mass after initiation of photo-polymerization. The rate of crosslinking reaction depends on the light intensity and the duration of the exposure. For example (which example is intended to be illustrative and not restrictive), exposure to the operating room light may be sufficient to cause the macromer some degree of cross-linking.

In another embodiment, polymerization may be carried out during production to form a flexible semisolid allograft. In another embodiment previously described, an injectable and moldable allograft putty of macromer, DBM and CCC may be formulated, but contains no crosslinking agent (such as a photoinitiator) and accordingly is not polymerized into a semisolid mass because of the lack of such agents.

In another embodiment, where PEG (polyethylene glycol) is employed as the water-soluble central block, the average molecular weight of PEG used in the macromer may be, for example (which example is intended to be illustrative and not restrictive), 20,000 Daltons. For each PEG in the macromer, there may be about 12 TMC (trimethylene carbonate) units and 4 LA (lactate) units that form a tripolymer with PEG. The ends of the PEG/TMC/LA tripolymer may be capped with acrylic ester end groups.

Macromers suitable for use as carriers, their methods of preparation, and their methods of use are disclosed in U.S. Pat. Nos. 5,900,245; 6,083,524; and 6,177,095, all of which are incorporated into the present disclosure by reference. Notably, however, the present applicants have found that the compositions described herein are effective without resort to the preparation and application of a primer composition that is disclosed in the 245 and 095 patents.

### EXAMPLES

### Example 1

3.377 grams of Glycerol (Aldrich) was blended into 2.1298 grams of demineralized bone matrix (TBI DBM lot # 990768 from Exactech, Gainesville Fla.) for a 61.4%/38.6% Glycerol/DBM ratio. The resulting putty was left at room temperature for 60 minutes and evaluated. The putty had an oily consistency and properties remained oily after a total of 3 hrs storage at room temperature.

### Example 2

3.4007 grams of Pluronic-127 solution (20% in DI water at 4° C.) was blended with 1.6046 grams of demineralized bone matrix (DBM) for a 67.9/32.1% Pluronic solution/DBM ratio. The resulting putty was left at room temperature for 3 hrs and evaluated for consistency. The putty was smooth and malleable when rolled into a small ball. No sign of cracking was observed when pressure was applied to squeeze out the ball shaped putty.

### Example 3

3.3635 grams of formulated FocalSeal®-S (FS-S) sealant macromer solution (10% concentration from Focal, Inc) was blended with 1.6061 grams of demineralized bone matrix (DBM) for a 67.7/32.3% FS-S/DBM ratio. The resulting putty was left at room temperature for 60 minutes and evaluated for consistency. The putty was smooth. Cohesive and malleable when rolled into a small ball. No sign of “dry edges” was observed when pressure was applied to squeeze out the ball shaped putty.

### Example 4

3.3654 grams of formulated FocalSeal®-S sealant macromer solution (10% concentration from Focal, Inc) was blended with 0.7022 grams of demineralized bone matrix (DBM) and 1.7988 grams of bone chips (from Exactech TBI lot # 12003476), resulting in the following ratio of FocalSeal®-S sealant/DBM/Bone Chips: 57.3%/12.0%/30.7%. The resulting putty was left at room temperature for 3 hrs. and evaluated for consistency. The putty was dry and cracked with dry edges when pressure was applied to squeeze out the ball shaped putty.

### Example 5

3.5865 grams of formulated FocalSeal®-S sealant macromer solution (10% concentration from Focal, Inc) was blended with 0.7002 grams of demineralized bone matrix (DBM) and 1.7922 grams of bone chips (from Exactech TBI lot # 12003476), resulting in the following ratio of FS—S/DBM/Bone Chips: 59.0%/11.5%/29.5%. The resulting putty was left at room temperature for 3 hrs. and evaluated for consistency. The putty was dry and cracked when pressure was applied to squeeze out the ball shaped putty, but showed improvements in its cohesiveness with a 2% FS-S increase as binder.

### Example 6

3.581 grams of formulated FocalSeal®-S sealant macromer solution (10% concentration from Focal, Inc) was blended with 1.5032 grams of demineralized bone matrix (DBM), resulting in the following ratio of FS—S/DBM: 70.0%/30.0%. The resulting putty was left at room temperature for 3 hours and evaluated for consistency. The putty was malleable and cohesive and did not form a dry edge when pressure was applied to squeeze out the ball shaped putty.

### Example 7

3.334 grams of formulated FocalSeal®-S sealant macromer solution (10% concentration from Focal, Inc) was blended with 0.5981 grams of DBM and 1.5056 grams of Bone Chips, resulting in the following ratio of FocalSeal®-S sealant/DBM/Bone Chips: 61.3%/11.0%/27.7%. The resulting putty was left at room temperature for 3 hrs. and evaluated for consistency. The putty was malleable and cohesive but showed dry edge when pressure was applied to squeeze out the ball shaped putty.

TABLE 1

| | % | % Bone | | | |
|---|---|---|---|---|---|
| | DBM | Chips | | | |
| Example | lot | lot | FS-S (10%) | Other Medium | |
| # | #990768 | #001037 | % | % | |
| 1 | 38.6 | 0 | 0 | Glycerol | 61.4 |
| 2 | 32.1 | 0 | 0 | Pluronic F-127 | 67.9 |
| 3 | 32.3 | 0 | 67.7 | NA | NA |
| 4 | 12 | 30.7 | 57.3 | NA | NA |
| 5 | 11.5 | 29.5 | 59 | NA | NA |
| 6 | 30 | 0 | 70 | NA | NA |
| 7 | 11 | 27.7 | 61.3 | NA | NA |

### Examples 8 through 12

Mixing and handling of 10% formulated FocalSeal®-S sealant with the indicated amounts in Table 2 of DBM ranging from 0 to 40% of solids. Approximately 0.7 grams to 0.85 grams of opaque formulation was delivered into a 15 mm ID×5 mm deep Teflon® mold and illuminated for 80 seconds with visible light to polymerize the composite. The gels were hydrated in Phosphate-buffer, pH=7.4, at 37° C. over approximately 16 days and % moisture uptake was measured.

TABLE 2

| | | % Hydration Days (hours) | | | | | | |
|---|---|---|---|---|---|---|---|---|
| Example | DBM (%) | 1 | 2 | 3 | 6 | 8 | 13 | 16 |
| # | in FS-S | (23) | (41) | (67) | (138) | (183) | (301) | (382) |
| 8 | 0 | 82.8 | 89.5 | 92.4 | 100.9 | 108.5 | 108.7 | 117.6 |
| 9 | 10 | 72.9 | 78.7 | 78.3 | 86.4 | 94.5 | 104.0 | 114.7 |
| 10 | 20 | 53.4 | 55.7 | 55.1 | 62.6 | 65.5 | 70.8 | 83.6 |
| 11 | 30 | 51.5 | 55.4 | 57.0 | 63.1 | 65.0 | 68.4 | 75.8 |
| 12 | 40 | 56.6 | 61.7 | 61.0 | 66.5 | 68.4 | 73.7 | 83.6 |

Handling observations of putties prior to polymerization.

10% DBM was the softest as to be expected, but was workable with a slightly sticky consistency. Material transfer for molding using a spatula.

20% DBM firmer and more even particle distribution due to a more dense formulation. Softer, slightly flowing properties. Material transfer for molding using a spatula.

30% DBM firm putty like material. Easily moldable, keeping its shape prior to polymerization

40% DBM firm putty like material. Firmer than 30% DBM and dry, moldable, keeping its shape, prior to polymerization

### Example 13

A 10% FocalSeal®-S sealant macromer (Focal, Inc. lot# 052300SF) solution was prepared in PBS. 2.1671 grams of this macromer solution was blended with 0.8960 grams of DBM (from Exactech, TBI lot # 990768/19) and left at room temperature for 60 minutes. Approximately 12×50 mg samples were placed into a petri dish and the putty lyophilized. The resulting dry composite was removed from petri dish and wetted with few drops of DI water, rolled into a little ball and allowed to hydrate further by the addition of a few more drops of water until a desired consistency was achieved. The putty was cohesive and malleable.

### Example 14

1.5015 g DBM and 0.3499 g of dry 20KTLA2 macromer powder was weighed into a 15 mL Nalgene container, followed by 3.5 mL of PBS Buffer. The components were blended in a capped jar using a spatula and allowed sit at room temperature for five minutes to fully hydrate the macromer. The resulting bone putty was then mixed further physically using gloved hands. The bone putty was very cohesive and kept its shape when rolled into a ball. No gel particles of hydrated macromer were noticed.

### Example 15

To show that example 14 may be made into a photopolymerizable graft, the following experiment was conducted:

Bone putty from example 14 was further blended with 0.6 mL of PBS buffer concentrate (containing approximately 0.054 triethanolamine, 0.08 g potassium phosphate and 40 ppm Eosin Y per total graft). The buffer concentrate was blended into the graft until an evenly pink colored putty was obtained. The putty was illuminated with visible light for 40 seconds, to induce photopolymerization of the macromer (450-550 nm, Xenon light source). The putty was then turned and illuminated for an additional 40 seconds on the other side to repeat the polymerization process. The resulting graft was malleable hydrogel and kept its shape.

### Example 16

Other manners of polymerization may be used for grafts containing DBM. For example (which example is intended to be illustrative and not restrictive), polymerization may be initiated by thermal initiation. A 0.700 g solution of macromer with 0.147 g solids, containing 5.88 mg of benzoyl peroxide, was prepared. Then 0.1039 g (10.4% by weight) of bone chips with a particle size of >0.5-<1.18 mm, and 0.1959 g (19.6% by weight) of DBM (demineralized bone material) with a particle size of <0.5 mm was incorporated into the solution. The resulting thick slurry was shaped into a 12 mm×2.5 mm disc, frozen and lyophilized. Once lyophilized, crosslinking of macromer in the shaped disks was initiated at 50° C. over a 10-hour time period under vacuum. The resulting material had formed a single, cohesive flexible matrix. The matrix was able to be re-hydrated in water and was easily manipulated without fragmenting or disrupting. Re-drying and rewetting of the DBM/Bone-chip/Hydrogel matrix at room temperature was feasible.

### Example 17

To determine if human DBM retained its osteoinductive ability when formulated with a macromer carrier, the following study was conducted.

Human DBM provided by an AATB accredited tissue bank, Tissue Banks International (TBI, Batch No. SF9904005045, San Rafael, Calif.) was aseptically processed and freeze-dried. The average particle size of DBM was in the range of 125 to 1000 μm. The sterile carrier provided by Focal, Inc. (Lexington, Mass.) was a polyethylene glycol based macromer with molecular weight of 20,000. DBM powders were mixed with a 10 wt % macromer solution in sterile phosphate buffer at three concentrations: 20, 30 and 40% by weight. Controls included TBI DBM alone and macromer carrier alone. All materials were pre-loaded into sterile gelatin capsules (size #5, Batch No. 07.039.90, Torpac, Inc. Fairfield, N.J.) (15 mg sample/capsule) and stored at −20° C. until surgery.

Five mice with compromised immune systems were used for each variable (nu/nu mice; Harlan Labs, Indianapolis Ind.). Mice were acclimated in the vivarium for 5 days prior to surgery. Each mouse received two identical implants, one in each calf muscle, resulting in 10 implants per variable. The surgery was conducted under protocol # 01056-34-01 B2) which was reviewed and approved by the Institutional Animal Care and Use Committee at the University of Texas Health Science Center at San Antonio (UTHSCSA).

Published studies using rat DBM indicate that osteoinduction occurs within 28 days. However, a number of studies using human DBM have found that osteoinduction occurs at a slower rate, and may not be evident at 28 days if at all. For this reason, many laboratories examine human DBM-implanted tissues at 35 days post-implantation or even later. Considerable variability in human DBM preparations have been shown, due in part to differences in processing as well as due to inter-donor variation. It has been found that many preparations that fail to exhibit osteoinduction ability at 28 days are osteoinductive at 56 days.

At 28 days post-surgery, implanted tissues were harvested from 1 mouse per variable to determine if carrier was resorbed and if there was an adverse tissue reaction. The tissue was fixed in buffered formalin and shipped to Northeast Ohio Universities College of Medicine for peripheral quantitative computed tomography (pQCT) bone mineral analysis. These tissues were subsequently returned to San Antonio for histology.

At 56 days post-surgery, the remaining 4 mice per variable were euthanized. The implanted tissues were harvested and x-rayed. Harvested tissues were processed for routine light microscopy and histologic analysis. Paraffin sections were stained with haematoxylin and eosin.

The osteoinduction ability of the materials was determined as described in the ASTM F04.47.01 “Draft Guidance on In Vivo Testing for Osteoinduction Ability.” For each implant, scoring was done on a single representative section. The section was selected as having the largest surface area, ideally from the center of the implanted tissue. The tibia and fibula were used to orient the reviewer, since both bones were present in the cross section. If the cross-section of both bones was not present, or if they had an elliptical appearance, the section was rejected. This requirement also allowed the reviewer to assure himself that any ossicles were due to the implant and not to the bones.

The following scoring system was used:

0 No DBM and no ossicles

1 DBM only

2 DBM plus one new ossicle

3 DBM plus two new ossicles

4 DBM plus ossicles covering the entire section

Results

At 28 days post surgery, pQCT indicated that all three formulations were osteoinductive since the scans were positive for mineral. However, histologic analysis of the specimens failed to show the presence of bone except one 20% DBM test sample, suggesting that the pQCT revealed the presence of remineralized DBM. All macromer carriers were completely resorbed by 28 days. There was no evidence of pathology in any of the implanted tissues indicating that the polyethylene glycol based macromer carrier was biocompatible.

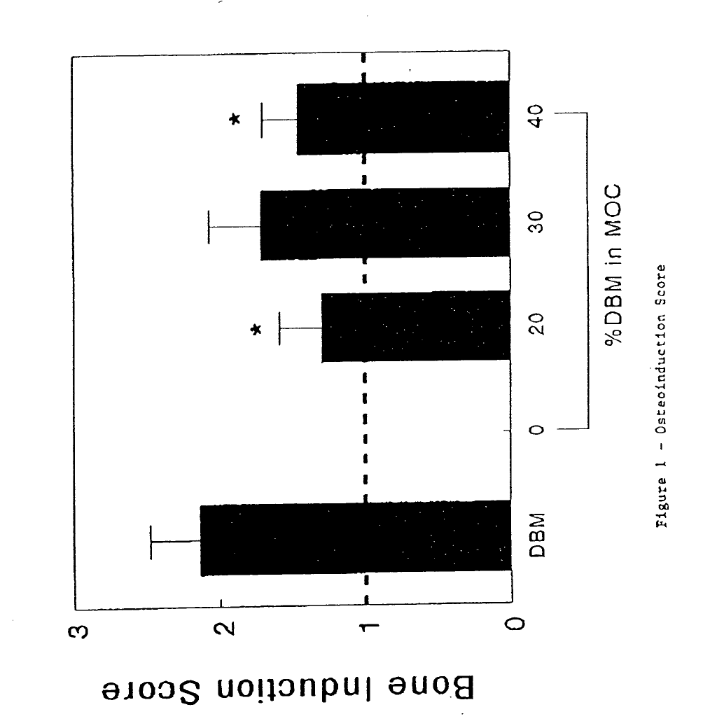

At 56 days, the TBI DBM and the DBM/macromer formulations were osteoinductive (FIG. 1). There was no difference in the osteoinduction ability of the TBI DBM and the 30% DBM test group, indicating the formulation containing 30% DBM was as effective as the TBI DBM control.

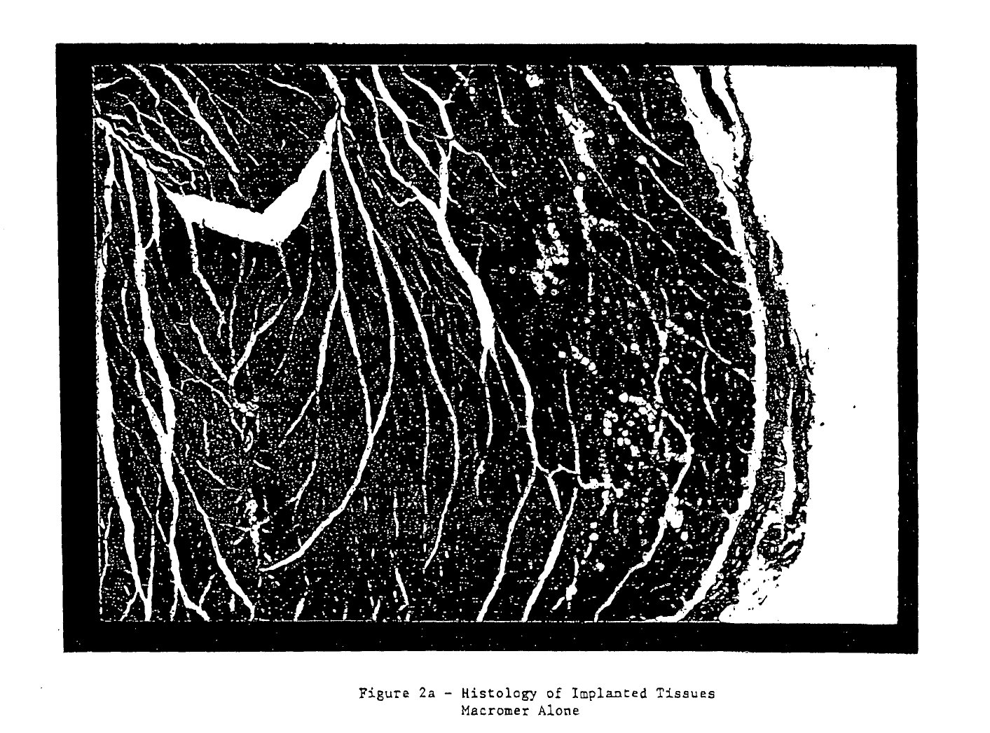

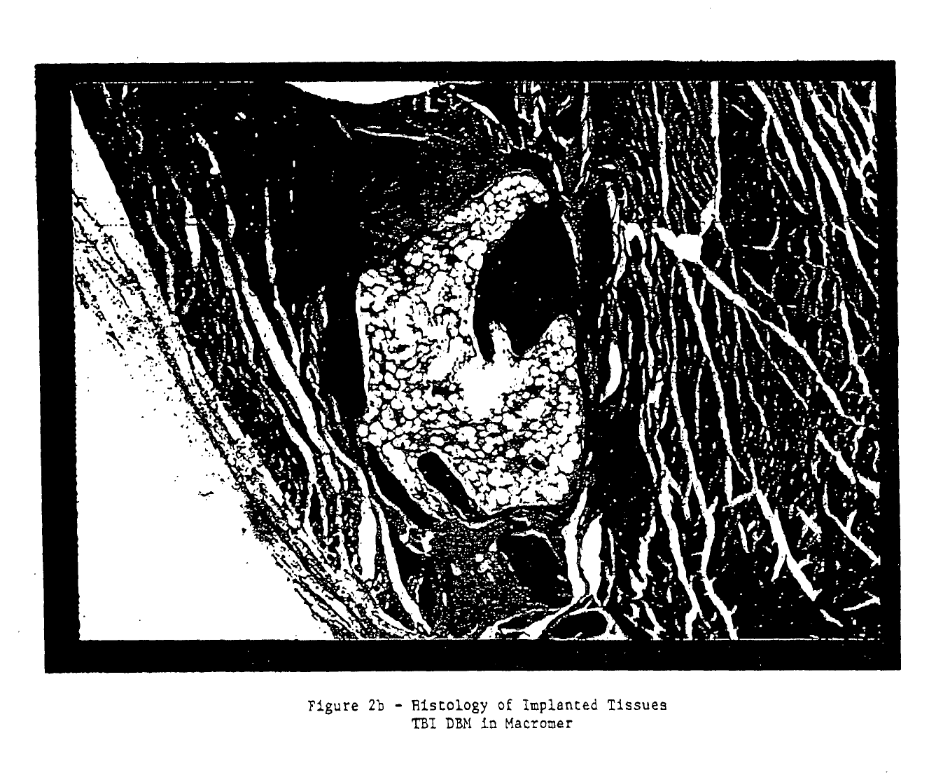

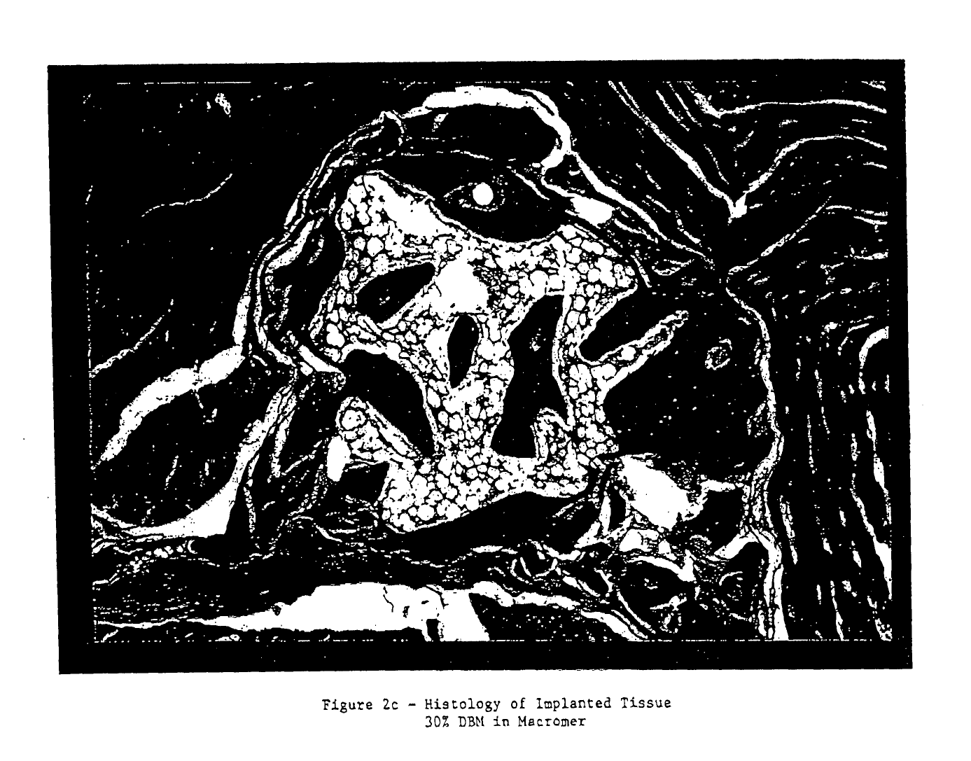

All implanted tissues were normal (FIGS. 2a, 2b, 2c). There was no evidence of any adverse tissue response, regardless of the implant used. Bone ossicles were typical in appearance, with a rim of cortical bone surrounding the bone trabeculae and haematopoietic bone marrow. In all instances, the macromer was completely resorbed, regardless of treatment.

Discussion and Conclusions

The results show that the macromer used in this example is a safe and effective carrier of DBM. The carrier is resorbed, causing no adverse reaction in the implanted tissue, and does not prevent the osteoinduction by human DBM. The optimal concentration of DBM was 30%. This probably is due to the specific packing characteristics of the bone powder in the carrier. However, 20% and 40% DBM formulations were also osteoinductive at 56 days, and one 20% DBM sample was able to induce new bone formation at 28 days. Osteoinduction in mice receiving 20% and 40% DBM implants was comparable to that observed in mice in the 30% DBM test group, although it was not as high as observed in the control mice. This suggests that the 20%40% range is acceptable, especially when using DBM preparations with very high osteoinduction ability. The TBI DBM used to make the formulations had not been tested previously, so it was not known prior to the study if it was indeed osteoinductive on its own.

### Example 18

### Posterolateral Fusion with a Novel Resorbable Polymer: Evaluation in a Rabbit Model

Introduction: Although autograft bone remains the gold standard graft material for spinal fusion, morbidity after graft harvest remains a concern. Frozen allograft bone offers one alternative to fresh autograft, but its use is associated with unpredictable clinical results and potential problems with disease transmission. A safe and effective alternative to allograft bone is needed. Ideally, this material would produce fusion rates that are equivalent to those seen with autograft. In reality, it may be more realistic to use the material as a bone graft extender to optimize fusion rates in patients with either limited supplies of autograft, or autograft that is poorly osteotherapeutic. To this end, a new bone graft substitute material has been developed by combining a novel resorbable polymer carrier (Macromer; Genzyme Biosurgery, Lexington, Mass.) with demineralized bone matrix (DBM). The specific aims of this study were (1) to confirm that the polymer-DBM product is osteoinductive in vivo and (2) to determine whether the new graft substitute is effective as either a standalone graft material or a bone graft extender in posterolateral fusion.

Methods: Eighteen male New Zealand White rabbits underwent bilateral intertransverse process fusion at L5-L6 using published techniques. All surgical procedures were reviewed and approved by the Institutional Animal Care and Use Committee. The fusion site was grafted with either autogenous corticocancellous bone (n=6), Macromer containing rabbit DBM (n=6) or Macromer-DBM in combination (1:1) with either autograft or allograft rabbit bone (n=3 per group).

To assess osteoinductivity, intramuscular implants containing DBM powder, a hydrated form of Macromer-DBM (Wet Macromer-DBM) or a lyophilized form of Macromer-DBM (Dry Macromer-DBM) were placed bilaterally in the quadriceps muscles of 9 rabbits (n=6 samples per implant).

Animals were euthanized 5 weeks post-surgery. Muscle specimens were excised and radiographed in a microradiography cabinet. If mineralization was identified, muscle specimens were fixed in alcohol and processed for undecalcified histology to confirm the presence of heterotopic ossification. The lumbar spine was harvested en bloc and radiographed in two planes (anterior-posterior and lateral). Specimens for mechanical testing were cleaned of all musculature and vessels. The facet joints at the operated level were removed with rongeurs, and the intervertebral disc divided with a scalpel so that the L5 and L6 vertebra were connected only by the posterolateral fusion mass. The L6 vertebra was potted in dental cement, and the L5 vertebra was transfixed with a metal pin that attached to a non-constrained fixture in the MTS frame. Nondestructive mechanical tests were then performed under load control, with load-displacement data being recorded continuously. Stiffness data were calculated between 60-120 N of load for the last three cycles and the results averaged for each specimen.

Radiographic data were analyzed by Chi-square analysis. Biomechanical data were analyzed by one-way analysis of variance (ANOVA). A significance level of p<0.05 was used for all analyses.

Results: Recovery after surgery was generally excellent in these animals. There were no complications associated with the use of the graft material either on its own or in combination with autograft or allograft.

The Macromer-DBM mixture was found to be osteoinductive within muscle. Radiographic evidence of mineralization was seen in all of the sites implanted with the wet and dry formulations (Table 3) Mineralization was also seen in the positive controls (muscles implanted with rabbit DBM powder). Histological examination confirmed the presence of viable new bone formation and active remodeling within the graft site. As anticipated from previous published work, radiographic evidence of fusion was seen in approximately 60% of the autograft controls. All of the graft alternatives performed at least as well as autograft (Table 4), although the differences did not reach statistical significance (p>0.05 for all comparisons).

TABLE 3
Microradiographic evidence of mineralization within
intramuscular sites implanted with DBM and Macromer-DBM.

| Graft Material | Mineralization Rate |
|---|---|
| DBM Only | 6/6 |
| Wet Macromer-DBM | 6/6 |
| Dry Macromer-DBM | 6/6 |

TABLE 4
Radiographic evidence of fusion at the L5-L6 intertransverse
space. The left and right sides were assessed independently
in each animal.

| Graft Material | Fusion Rate |
|---|---|
| Autograft | 7/12 (58%) |
| Macromer-DBM | 9/12 (75%) |
| Macromer-DBM-Autograft | 5/6 (83%) |
| Macromer-DBM-Allograft | 4/6 (66%) |

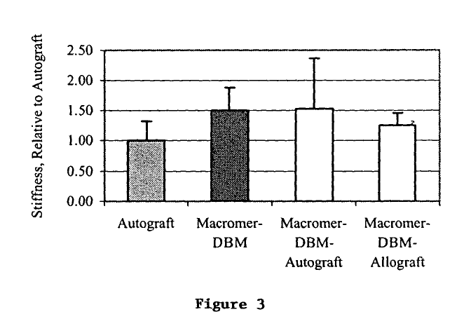

Biomechanical test data are presented in FIG. 3 (showing Mechanical test results; Data represent mean (SD) stiffness for n6 specimens per group (n=3 for Macromer-DBM-Autograft and Macromer-DBM-Allograft)). As with the radiographic data, the graft alternatives performed at least as well as the autograft controls in this model. Widespread scatter in the data made it difficult to achieve acceptable statistical power, even with a group size of n=6 per treatment, but the Macromer-DBM group showed a strong trend towards higher stiffness values as compared to the autograft controls (p=0.083).

Discussion: The use of the resorbable polymer carrier, Macrorner, in combination with DBM appears to produce radiographic and mechanical test results that are at least equivalent to those of the gold standard autograft control. Given the inherent difficulty in establishing that a new treatment is “significantly better” than autograft, these preliminary data in an established animal model are extremely encouraging. Continued investigation into the use of this material, as either an alternative to autograft or as a graft extender, is warranted. Ultimately, the use of an off-the-shelf bone graft substitute with proven efficacy should translate into improved outcomes for patients that are undergoing spine fusion procedures.

### Example 19

### Evaluation of a Demineralized Bone Matrix in a Nude Rat Tibial Defect Model

Introduction: Demineralized Bone Matrix (DBM) has been shown to be beneficial for bone regeneration and has become accepted as a clinical bone-graft substitute in variety of skeletal sites. Bone induction using DBM has been traditionally studied in non-skeletal sites. However, several studies have questioned the inductive capacity of DBM. Lack of inductive properties of DBM may be a result of preparation and sterilization. This pilot study is an evaluation of the bone inductive capabilities in a bony site of commercial DBM preparations using a nude rat tibial defect model.

Methods: Male athymic NIH-RNU (nude) rats aged 11-12 weeks old (National Cancer Institute, MA) were used following ethical approval. A critical size defect (8 mm long×3 mm wide) was created on the anteromedial surface of the tibia, distal to the MCL attachment. The posterior and anterolateral cortices were preserved. The defects were filled with DBM (Exactech, Inc., FL) (n=4 per group) (Table 5; groups 3-9). Autograft and empty defect groups were included as positive and negative controls. Animals were euthanased at 1 and 3 weeks and the entire intact tibia was x-rayed, mechanically tested in cantilever bending (3-week samples only). Tibias were fixed in formalin, decalcified in formic acid, sectioned and stained with H&E. Histology was graded in centre of the defect in a blinded fashion by 3 reviewers. Mechanical data was analyzed using a 1-way ANOVA (SPSS for Windows).

TABLE 5
Study Groups

| Group | Treatment |
|---|---|
| 1 | Empty Defect |
| 2 | Auto graft |
| 3 | Carrier |
| 4 | DBM + carrier |
| 5 | Freeze dried DBM + carrier |
| 6 | Light activated DBM + carrier |
| 7 | DBM |
| 8 | Inactive DBM + carrier |
| 9 | InactiveDBM |

Essential Results: Radiographs confirmed the empty defects were not healed. Variability in radiographic appearance in groups 4-9 was noted. Mechanical testing at 3 weeks revealed the autograft group to have a higher fracture load, but was not statistically significant. The stiffness in the autograft group was greater compared to all other groups (p<0.05).

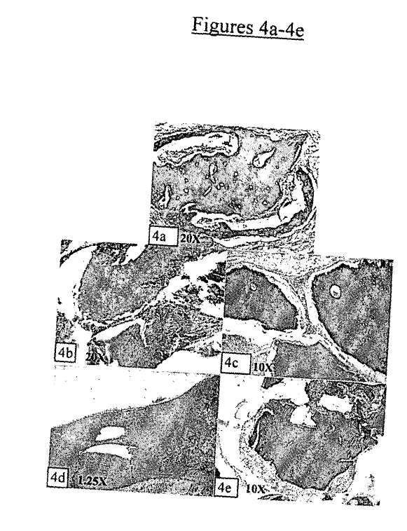

Histology did not show any new bone formation at week 1 in the DBM treated defects (groups 4, 5, 6). New bone formation was evident by week 3 in the DBM treated defects (FIG. 4a). New bone formation was found at week 1 in defects filled with autograft (FIG. 4b). New bone formation was not observed at any time point in the inactive DBM groups (FIG. 4c), empty defects (FIG. 4d) or carrier alone. Results indicated that the light activated samples had stronger inductive capabilities with evidence of endochondral ossification at week 3 (FIG. 4e). The presence of the carrier either alone or in combination with DBM (active or inactive) did not present any early adverse reaction. Residual demineralized bone with the characteristic acellular appearance was noted in the defects at 1 and 3 weeks with little resorption and evidence of osteoclastic activity.

Discussion: The use of demineralized bone has a long clinical history since it was reported by Urist. DBM contains a number of osteoinductive proteins known to be involved in bone formation as well as providing a potential new matrix. This may have significant benefits over the use of a single osteoinductive protein. Variability in the invivo response to DBM has been reported histologically and was confirmed in this pilot study in a skeletal site.

Controls, DBM and inactivated DBM performed as expected. The mechanical testing protocol developed in this study applied a tensile load to the superior aspect of the defect and demonstrated the autograft to be stiffer. This agrees with the histological observations of new bone formation at 1 and 3 weeks. These results, while preliminary in nature, support the use of a nude rat skeletal model for osteoinduction of DBM and carriers.

While a number of embodiments of the present invention have been described, it is understood that these embodiments are illustrative only, and not restrictive, and that many modifications may become apparent to those of ordinary skill in the art.

What is claimed is:

1. A composition, comprising:
a carrier; and
an osteotherapeutic material;
wherein the carrier is a macromer comprising: (a) a water-soluble block; and (b) at least one of: (i) a biodegradable block, wherein the biodegradable block comprises a linkage based on a carbonate or ester group; and (ii) a polymerizable group.

2. The composition of claim 1, wherein the composition is in the form of one of: (a) an aqueous mixture; and (b) a non-hydrated form.

3. The composition of claim 2, wherein the osteotherapeutic material is selected from the group of: (a) demineralized bone matrix; and (b) cortical-cancellous bone chips.

4. The composition of claim 2, wherein the osteotherapeutic material is selected from the group of: (a) an osteoinductor; (b) an osteoconductor; (c) an osteogenic factor; and (d) an osteopromoter.

5. The composition of claim 2, wherein the composition is resorbed and replaced by new bone substantially throughout the volume of the composition after implantation in a vertebrate.

6. The composition of claim 5, wherein a ratio of the carrier to the osteotherapeutic material is selected to provide an effective amount of each such that the composition is resorbed and replaced by new bone substantially throughout the volume of the composition.

7. The composition of claim 5, wherein the osteotherapeutic material is provided in an effective amount such that the composition is resorbed and replaced by new bone substantially throughout the volume of the composition.

8. The composition of claim 5, wherein the vertebrate is a mammal.

9. The composition of claim 8, wherein the mammal is a human.

10. The composition of claim 2, further including an initiator for inducing a polymer forming reaction with the polymerizable group.

11. The composition of claim 10, wherein the initiator is included in the carrier.

12. The composition of claim 10, wherein the initiator is selected from the group of: (a) a photo initiator; (b) a thermal initiator; and (c) a chemical initiator.

13. The composition of claim 12, wherein the photo initiator is Eosin Y.

14. The composition of claim 12, wherein the chemical initiator is a peroxide.

15. The composition of claim 2, wherein polymerization is initiated by a reaction selected from the group of: (a) photopolymerization; (b) chemical free-radical polymerization; (c) thermal free-radical polymerization; (d) redox reaction; (e) cationic polymerization; and (f) chemical reaction of active groups.

16. The composition of claim 2, wherein the carrier is further comprised of a free radical generating combination of a transition metal, a peroxide, and a stabilizing agent.

17. The composition of claim 2, wherein the macromer comprises at least one of: (a) poly(ethylene glycol); (b) trimethylene carbonate moieties; (c) lactic acid ester moieties; (d) acrylic ester moieties; and (e) combinations thereof.

18. The composition of claim 2, further comprising an additive to modify at least one of a physical and a chemical aspect of the composition.

19. The composition of claim 2, further comprising an additive to modify a biological aspect of the composition.

20. The composition of claim 1, wherein the water soluble block is selected from the group of poly(ethylene glycol) and poly(ethylene oxide).

21. The composition of claim 1, wherein the biodegradable block includes polymers and oligomers of hydroxy acids.

22. The composition of claim 1, wherein the ester group includes hydroxy acid ester moieties selected from the group of glycolic acid, DL-lactic acid and L-lactic acid.

23. The composition of claim 1, wherein the carbonate group is selected from a group derived from at least one of trimethylene carbonate and dimethyl carbonate.

24. The composition of claim 1, wherein the polymerizable group contains at least one of: (a) a macromer-macromer functional group that reacts spontaneously or under the influence of light, heat or other activating conditions or reagents to form a covalent polymeric structure that binds strands of the macromer to one another; and (b) a reactive functional group for converting a solution of the macromer into a gel.

25. The composition of claim 24, wherein the macromer-macromer functional group is selected from the group of: (a) ethylenic groups; (b) epoxides; (c) lactams; and (d) latones.

26. The composition of claim 24, wherein the reactive functional group is selected from the group of: (a) activated esters; (b) electrophilic carbon centers; (c) conjugated ethylenic groups; (d) isocyanates; (e) isothiocyanates; (f) oxirane; (g) aziridines; (h) cyclic imides; (i) sulfhydryls; and (j) combinations thereof.

27. A method of manufacturing a composition, comprising:
mixing a carrier and an osteotherapeutic material;
wherein the carrier is a macromer comprising: (a) a water-soluble block; and (b) at least one of: (i) a biodegradable block, wherein the biodegradable block comprises a linkage based on a carbonate or ester group; and (ii) a polymerizable group.

28. The method of claim 27, wherein the composition is in the form of one of: (a) an aqueous mixture; and (b) a non-hydrated form.

29. The method of claim 28, wherein the osteotherapeutic material is selected from the group of: (a) demineralized bone matrix; and (b) cortical-cancellous bone chips.

30. The method of claim 28, wherein the osteotherapeutic material is selected from the group of: (a) an osteoinductor; (b) an osteoconductor; (c) an osteogenic factor; and (d) an osteopromoter.

31. The method of claim 28, wherein the composition is resorbed and replaced by new bone substantially throughout the volume of the composition after implantation in a vertebrate.

32. The method of claim 31, wherein a ratio of the carrier to the osteotherapeutic material is selected to provide an effective amount of each such that the composition is resorbed and replaced by new bone substantially throughout the volume of the composition.

33. The method of claim 31, wherein the osteotherapeutic material is provided in an effective amount such that the composition is resorbed and replaced by new bone substantially throughout the volume of the composition.

34. The method of claim 31, wherein the vertebrate is a mammal.

35. The method of claim 34, wherein the mammal is a human.

36. The method of claim 28, further comprising including in the composition an initiator for inducing a polymer forming reaction with the polymerizable group.

37. The method of claim 36, further comprising including the initiator in the carrier.

38. The method of claim 36, wherein the initiator is selected from the group of: (a) a photo initiator; (b) a thermal initiator; and (c) a chemical initiator.

39. The method of claim 38, wherein the photo initiator is Eosin Y.

40. The method of claim 38, wherein the chemical initiator is a peroxide.

41. The method of claim 28, further comprising applying radiation to the carrier.

42. The method of claim 28, further comprising initiating polymerization by a reaction selected from the group of: (a) photopolymerization; (b) chemical free-radical polymerization; (c) thermal free-radical polymerization; (d) redox reaction; (e) cationic polymerization; and (f) chemical reaction of active groups.

43. The method of claim 28, wherein the carrier is further comprised of a free radical generating combination of a transition metal, a peroxide, and a stabilizing agent.

44. The method of claim 28, wherein the macromer comprises at least one of: (a) poly(ethylene glycol); (b) trimethylene carbonate moieties; (c) lactic acid ester moieties; (d) acrylic ester moieties; and (e) combinations thereof.

45. The method of claim 28, further comprising including in the composition an additive to modify at least one of a physical and a chemical aspect of the composition.

46. The method of claim 28, further comprising including in the composition an additive to modify a biological aspect of the composition.

47. The method of claim 27, wherein the water soluble block is selected from the group of poly(ethylene glycol) and poly(ethylene oxide).

48. The method of claim 27, wherein the biodegradable block includes polymers and oligomers of hydroxy acids.

49. The method of claim 27, wherein the ester group includes hydroxy acid ester moieties selected from the group of glycolic acid, DL-lactic acid and L-lactic acid.

50. The method of claim 27, wherein the carbonate group is selected from a group derived from at least one of trimethylene carbonate and dimethyl carbonate.

51. The method of claim 27, wherein the polymerizable group contains at least one of: (a) a macromer-macromer functional group that reacts spontaneously or under the influence of light, heat or other activating conditions or reagents to form a covalent polymeric structure that binds strands of the macromer to one another; and (b) a reactive functional group for converting a solution of the macromer into a gel.

52. The method of claim 51, wherein the macromer-macromer functional group is selected from the group of: (a) ethylenic groups; (b) epoxides; (c) lactams; and (d) latones.

53. The method of claim 51, wherein the reactive functional group is selected from the group of: (a) activated esters; (b) electrophilic carbon centers; (c) conjugated ethylenic groups; (d) isocyanates; (e) isothiocyanates; (f) oxirane; (g) aziridines; (h) cyclic imides; (i) sulfhydryls; and (j) combinations thereof.

54. A method of treating a bone defect in a patient, comprising:
implanting in the patient at the site of a defect a composition comprising a carrier and an osteotherapeutic material;
wherein the carrier is a macromer comprising: (a) a water-soluble block; and (b) at least one of: (i) a biodegradable block, wherein the biodegradable block comprises a linkage based on a carbonate or ester group; and (ii) a polymerizable group.

55. The method of claim 54, wherein the composition is in the form of one of: (a) an aqueous mixture; and (b) a non-hydrated form.

56. The method of claim 55, wherein the osteotherapeutic material is selected from the group of: (a) demineralized bone matrix; and (b) cortical-cancellous bone chips.

57. The method of claim 55, wherein the osteotherapeutic material is selected from the group of: (a) an osteoinductor; (b) an osteoconductor; (c) an osteogenic factor; and (d) an osteopromoter.

58. The method of claim 55, wherein the composition is resorbed and replaced by new bone substantially throughout the volume of the composition after implantation in a vertebrate.

59. The method of claim 58, wherein a ratio of the carrier to the osteotherapeutic material is selected to provide an effective amount of each such that the composition is resorbed and replaced by new bone substantially throughout the volume of the composition.

60. The method of claim 58, wherein the osteotherapeutic material is provided in an effective amount such that the composition is resorbed and replaced by new bone substantially throughout the volume of the composition.

61. The method of claim 58, wherein the patient is a mammal.

62. The method of claim 61, wherein the mammal is a human.

63. The method of claim 55, further comprising including in the composition an initiator for inducing a polymer forming reaction with the polymerizable group.

64. The method of claim 63, further comprising including the initiator in the carrier.

65. The method of claim 63, wherein the initiator is selected from the group of: (a) a photo initiator; (b) a thermal initiator; and (c) a chemical initiator.

66. The method of claim 65, wherein the photo initiator is Eosin Y.

67. The method of claim 65, wherein the chemical initiator is a peroxide.

68. The method of claim 55, further comprising applying radiation to the carrier.

69. The method of claim 55, further comprising the step of polymerization, which polymerization is initiated by a reaction selected from the group of: (a) photopolymerization; (b) chemical free-radical polymerization; (c) thermal free-radical polymerization; (d) redox reaction; (e) cationic polymerization; and (f) chemical reaction of active groups.

70. The method of claim 55, wherein the carrier is further comprised of a free radical generating combination of a transition metal, a peroxide, and a stabilizing agent.

71. The method of claim 55, wherein the macromer comprises at least one of: (a) poly(ethylene glycol); (b) trimethylene carbonate moieties; (c) lactic acid ester moieties; (d) acrylic ester moieties; and (e) combinations thereof.

72. The method of claim 55, further comprising including in the composition an additive to modify at least one of a physical and a chemical aspect of the composition.

73. The method of claim 55, further comprising including in the composition an additive to modify a biological aspect of the composition.

74. The method of claim 54, wherein the water soluble block is selected from the group of poly(ethylene glycol) and poly(ethylene oxide).

75. The method of claim 54, wherein the biodegradable block includes polymers and oligomers of hydroxy acids.

76. The method of claim 54, wherein the ester group includes hydroxy acid ester moieties selected from the group of glycolic acid, DL-lactic acid and L-lactic acid.

77. The method of claim 54, wherein the carbonate group is selected from a group derived from at least one of trimethylene carbonate and dimethyl carbonate.

78. The method of claim 54, wherein the polymerizable group contains at least one of: (a) a macromer-macromer functional group that reacts spontaneously or under the influence of light, heat or other activating conditions or reagents to form a covalent polymeric structure that binds strands of the macromer to one another; and (b) a reactive functional group for converting a solution of the macromer into a gel.

79. The method of claim 78, wherein the macromer-macromer functional group is selected from the group of: (a) ethylenic groups; (b) epoxides; (c) lactams; and (d) latones.

80. The method of claim 78, wherein the reactive functional group is selected from the group of: (a) activated esters; (b) electrophilic carbon centers; (c) conjugated ethylenic groups; (d) isocyanates; (e) isothiocyanates; (f) oxirane; (g) aziridines; (h) cyclic imides; (i) sulfhydryls; and (j) combinations thereof.

81. The method of claim 54, further comprising the step of polymerization at the site of the bone defect.

82. The method of claim 81, further comprising the step of polymerization in an operating room.

83. The method of claim 54, further comprising the step of polymerization at a time of implantation.

84. The method of claim 54, further comprising the step of polymerization at a site remote from an operating room.

85. The method of claim 84, wherein the site remote from the operating room is a place of manufacture of the composition.

86. The method of claim 54, further comprising the step of polymerization at a time of manufacture of the composition.

87. The method of claim 54, further comprising the step of processing the composition by at least one process selected from the group of: (a) dessication; (b) dry blending; (c) lyophilization; and (d) granulation.

88. The method of claim 54, wherein the carrier is in a non-hydrated form and liquid is added to the non-hydrated form in an amount sufficient to form a solution of the carrier.

89. The method of claim 88, wherein the liquid is selected from the group of: (a) sterile water; (b) saline solution; (c) lactated ringer's solution; and (d) biological fluid.

90. The method of claim 88, wherein the liquid includes one or more components that aid in polymerization.

91. The method of claim 54, wherein the composition is in a non-hydrated form and liquid is added to the non-hydrated form in an amount sufficient to form a hydrated mixture of the composition.

92. The method of claim 91, wherein the liquid is selected from the group of: (a) sterile water; (b) saline solution; (c) lactated ringer's solution; and (d) biological fluid.

93. The method of claim 91, wherein the liquid includes one or more components that aid in polymerization.

94. The method of claim 54, wherein the composition takes the form selected from the group of: (a) a powder; (b) a dough; (c) a paste; (d) a solid; (e) a semi-sold; (f) granules; (g) a fiber; (h) a fabric; (i) a film; and (j) a monolithic.

95. The method of claim 54, further comprising the step of processing the composition by at least one process selected from the group of: (a) physical admixture; (b) covalent attachment; (c) ionic attachment; and (d) physical interpenetration.

96. The method of claim 54, further comprising the step of mixing the composition with fluid and then implanting.

97. The method of claim 54, further comprising the step of implanting the dry composition and then hydrating with a fluid.

98. The method of claim 54, further comprising the step of using the composition as a coating for an implant.

99. The method of claim 98, wherein the implant is selected from the group of: (a) a spinal cage; (b) a screw; (c) a knee/hip implant; (d) a periodontal implant; and (e) a craniofacial implant.

100. The method of claim 54, further comprising using the composition to grow bone outside the patient before implantation.

101. The method of claim 100, wherein the bone is grown outside the patient in a bioreactor.

102. A method of growing bone in a patient, comprising:
implanting at a heterotopic site in the patient a composition comprising a carrier and an osteotherapeutic material;
wherein the carrier is a macromer comprising: (a) a water-soluble block; and (b) at least one of: (i) a biodegradable block, wherein the biodegradable block comprises a linkage based on a carbonate or ester group; and (ii) a polymerizable group.

103. The method of claim 102, wherein the composition is in the form of one of: (a) an aqueous mixture; and (b) a non-hydrated form.

104. A composition, comprising:
a carrier; and
an osteotherapeutic material;
wherein the carrier is a macromer comprising: at least one water-soluble block; at least one biodegradable block, wherein the biodegradable block comprises a linkage based on a carbonate or ester group; and at least one polymerizable group.

105. The composition of claim 104, wherein the composition is in the form of one of: (a) an aqueous mixture; and (b) a non-hydrated form.

106. A method of manufacturing a composition, comprising:
mixing a carrier and an osteotherapeutic material;
wherein the carrier is a macromer comprising: at least one water-soluble block; at least one biodegradable block, wherein the biodegradable block comprises a linkage based on a carbonate or ester group; and at least one polymerizable group.

107. The method of claim 106, wherein the composition is in the form of one of: (a) an aqueous mixture; and (b) a non-hydrated form.

108. A method of treating a bone defect in a patient, comprising:
implanting in the patient at the site of the defect a composition comprising a carrier and an osteotherapeutic material;
wherein the carrier is a macromer comprising: at least one water-soluble block; at least one biodegradable block, wherein the biodegradable block comprises a linkage based on a carbonate or ester group; and at least one polymerizable group.

109. The method of claim 108, wherein the composition is in the form of one of: (a) an aqueous mixture; and (b) a non-hydrated form.

110. A method of growing bone in a patient, comprising:
implanting at a heterotopic site in the patient a composition comprising a carrier and an osteotherapeutic material;
wherein the carrier is a macromer comprising: at least one water-soluble block; at least one biodegradable block, wherein the biodegradable block comprises a linkage based on a carbonate or ester group; and at least one polymerizable group.

111. The method of claim 110, wherein the composition is in the form of one of: (a) an aqueous mixture; and (b) a non-hydrated form.
